SEQUENCE LISTING (20 sequences)

SEQ ID NO: 1 - DNA, 200 bp, Artificial
  a target polynucleotide for highly expressing PRRSV-ORF 7 in Escherichia coli
catatgccaaataacaacggcaagcagcagaagaaaaagaagggggacggccagccagtcaatcagctgtgccaaatgctgggtaagatcatcgcccagcaaagtcagtcccgtgttaagggtccggggcgtaaaaataagaagaaaaacccggagaagccgcattttcctctggcgactgaagatgacgtccgtcacca

SEQ ID NO: 2 - DNA, 30 bp, Artificial
  a forward primer
ccgcgcggcagccatatgccaaataacaac

SEQ ID NO: 3 - DNA, 36 bp, Artificial
  a reversed primer
cttcttatttttactacccggtcccttaactctgga

SEQ ID NO: 4 - DNA, 36 bp, Artificial
  a reversed primer
cttcttatttttacgacccggacccttaacacggga

SEQ ID NO: 5 - DNA, 35 bp, Artificial
  a reversed primer
tgcggcttctccgggtttttcttcttatttttacg

SEQ ID NO: 6 - DNA, 33 bp, Artificial
  a reversed primer
gtcgccagaggaaaatgcggcttctccgggttt

SEQ ID NO: 7 - DNA, 32 bp, Artificial
  a reversed primer
tggtgacggacgtcatcttcagtcgccagagg

SEQ ID NO: 8 - DNA, 162 bp, Artificial
  a target polynucleotide for highly expressing FMD-vpg in Escherichia coli
aaaaaatcccatatggagggtccatacgccggcccaatggagcgtcagaaaccgctgaaggtgaaagcaaaagccccggtcgtgaaggaaggtccttacgagggtccggtgaagaaacctgtcgctttgaaagtgaaagcaaagaacttgatcgtcactgag

SEQ ID NO: 9 - DNA, 123 bp, Artificial
  partial PET-23a plasmid
gtcgacaagcttgcggccgcactcgagcaccaccaccaccaccactgagatccggctgctaacaaagcccgaaaggaagctgagttggctgctgccaccgctgagcaataactagcataaccc

SEQ ID NO: 10 - DNA, 30 bp, Artificial
  a forward primer
ttgatcgtcactgaggtcgacaagcttgcg

SEQ ID NO: 11 - DNA, 30 bp, Artificial
  a reversed primer
ttatgctagttattgctcagcggtggcagc

SEQ ID NO: 12 - DNA, 31 bp, Artificial
  a forward primer
agtgaaagcaaagaacttgatcgtcactgag

SEQ ID NO: 13 - DNA, 31 bp, Artificial
  a forward primer
acctgtcgctttgaaagtgaaagcaaagaac

SEQ ID NO: 14 - DNA, 30 bp, Artificial
  a forward primer
ggtccggtgaagaaacctgtcgctttgaaa

SEQ ID NO: 15 - DNA, 30 bp, Artificial
  a forward primer
gaaggtccttacgagggtccggtgaagaaa

SEQ ID NO: 16 - DNA, 33 bp, Artificial
  a forward primer
aaagccccggtcgtgaaggaaggtccttacgag

SEQ ID NO: 17 - DNA, 34 bp, Artificial
  a forward primer
accgctgaaggtgaaagcaaaagccccggtcgtg

SEQ ID NO: 18 - DNA, 33 bp, Artificial
  a forward primer
ccaatggagcgtcagaaaccgctgaaggtgaaa

SEQ ID NO: 19 - DNA, 34 bp, Artificial
  a forward primer
gagggtccatacgccggcccaatggagcgtcaga

SEQ ID NO: 20 - DNA, 30 bp, Artificial
  a forward primer
aaaaaatcccatatggagggtccatacgcc

(19) United States
(12) Patent Application Publication
(10) Pub. No.: US 2009/0061488 A1
(43) Pub. Date: Mar. 5, 2009

(54) Method of synthesizing a target polynucleotide encoding a protein

(76) Inventors: Chao-Wei Liao, Chunan Town (TW); Shin-Hung Lin, Chunan Town (TW); Chi-Min Chen, Chunan Town (TW); Chung-Nan Weng, Chunan Town (TW)

Correspondence Address: LADAS & PARRY LLP, 26 WEST 61ST STREET, NEW YORK, NY, 10023, US

(21) Appl. No.: 11/796,739

(22) Filed: Apr. 27, 2007

Related U.S. Application Data
(63) Continuation-in-part of application No. 10/672,746, filed on Sep. 26, 2003, now abandoned.

(51) Int. Cl. C12P 19/34; C12N 15/64

(52) U.S. Cl. 435/91.2; 435/471

(57) ABSTRACT

The present invention provides a method of synthesizing a target polynucleotide encoding a protein, which uses a primer extension technique to constitute the target polynucleotide sequence. Preferably, the method is applied in a method for highly expressing a protein encoded by the target polynucleotide in a host.

### BACKGROUND OF THE INVENTION

1. Field of the Invention

The invention mainly relates to a method of synthesizing a target polynucleotide encoding a protein.

2. Description of the Related Art

Synthesis of a polynucleotide having a particular or given polynucleotide sequence is important to life science exploration. Such particular polynucleotide sequence usually encodes a protein, and especially a heterogeneous protein for expressing in a host cell. In conventional methods of synthesis of a known oligonucleotide having a particular sequence shorter than about 50 nucleotides, a chemical synthesis is used. However, it is difficult to synthesize a larger polynucleotide fragment through a chemical synthesis and the manipulation is complicated.

Generally, the polymerase chain reaction (PCR) is a usual method for amplifying a large polynucleotide fragment in vitro (Kleppe K. Ohtsuka E., Kleppe R., Molineux I. and Khorana H. G. 1971. Studies on polynucleotide. XCVI. Repair replication of short synthetic DNA is catalyzed by DNA polymerases. J. Mol. Biol. 56: 341-361). The PCR usually comprises four steps: (1) denaturalizing a template to form two single strands; (2) annealing two primers to the two strands in step (1), respectively; (3) extending the primers by DNA polymerase; and (4) obtaining two double strands of DNAs. Repeat the steps mentioned above, and a particular DNA fragment is amplified. To conduct a PCR, the following materials are needed: (a) a template which comprises a DNA fragment of the particular DNA sequence to be produced; (b) a pair of primers which hybridize the two strands of the template at the 5′-ends of the two strands, respectively; (c) DNA polymerase(s) and dNTP for synthesizing under proper conditions.

Some methods of the PCR are suitable for generating a polynucleotide having a little modifications relative to a template thereof such as a site-directed mutagenesis through a PCR (Ho S, N., Hunt H. D., Horton R. M., Pullen J. K., and Pease L. R. 1989. Site-directed mutagenesis by overlap extension using the polymerase chain reaction. Gene 77: 51-59). In the site-directed mutagenesis through a PCR, one or more nucleotides can be designed in a primer for being added, substituted or deleted in a wild type template sequence. However, if there are many mutation sites generated in one primer, the PCR cannot be successfully performed. The reason would be that a primer with many mutation sites leads a non-specific hybridization with the template. In a conventional PCR for generating many mutation sites in a small region, such as within 60 base pairs, a first stage of the PCR is conducted to generate some mutation sites with the use of the wild type sequence as a template. A product of the first stage of the PCR should be purified and then used as a template in a second stage of the PCR to generate some mutation sites on the product of the first stage of the PCR. A product of the second stage of the PCR should be purified and then used as a template in a third stage of the PCR to generate other mutation sites on the product of the second stage of the PCR. In most stages of the PCR, it may be necessary to generate mutation sites to the wild type sequence. Therefore, the manipulation is complicated and laborious.

Besides, a template molecule that is highly homologous to the product is required. However, if no original template can be obtained in some situations, there is no way to obtain the product by a conventional PCR.

In another aspect, some heterogeneous proteins cannot be expressed in a host-vector system because of the difference in codon usage between the protein original species and the host species. For example, a codon for tryptophan in human mitochondria or Mycoplasma spp. is UGA, but it is a stop signal in an Escherichia coli host-vector system. Therefore, in most cases, the protein expression yield is very low or unsatisfactory. Changing the codons seems to be a possible solution to the problem. For that matter, such codon change needs to generate multiple point mutation sites interspersed in a polynucleotide coding the heterogeneous protein comparing with the original gene obtained. However, for the above-mentioned reasons, it is difficult to obtain a desired result.

Therefore, an efficient and accurate method of synthesizing a target polynucleotide efficiently expressed in a host-vector expression system is still needed.

### SUMMARY OF THE INVENTION

The present invention provides a method of synthesizing a target polynucleotide encoding a protein through a polymerase chain reaction (PCR). Preferably, the method is applied in a method for highly expressing a protein encoded by the target polynucleotide in a host. The method preferably further comprises adjusting a sequence to change a codon of the target polynucleotide to a codon which has a high expression efficiency in translating a corresponding amino acid in a cell of the host.

One subject of the invention is to provide a method for synthesizing a target polynucleotide encoding a protein comprising conducting multi-cyclic polymerase chain reactions by a primer extension technique to obtain a product comprising the target polynucleotide; wherein a first polymerase chain reaction of the multi-cyclic polymerase chain reactions is conducted on a template and with a first set of primer pairs, and succeeding polymerase chain reactions are conducted on a template that is a product obtained in a previous polymerase chain reaction and the succeeding polymerase chain reactions are conducted with one or more sets of primer pairs comprising
(i) a second set of primer pairs comprising a second forward primer and a second reversed primer, the second forward primer having two parts:
(a) part (a1), located at the 5′-end region of the second forward primer, comprising a fragment having more than 10 nucleotides for forward extending the product obtained in the previous polymerase chain reaction and producing the target polynucleotide, and
(b) part (b1), located at the 3′-end region of the second forward primer, comprising a fragment having more than 10 nucleotides capable of annealing the second forward primer to the template and wherein the 3′-end of the part (a1) is adjacent to the 5′-end of the part (b1); and
the second reversed primer, located at the 3′-end region of the second reversed primer, comprising a fragment having more than 5 nucleotides capable of annealing the second reversed primer to the template;
(ii) a third set of primer pairs comprising a third forward primer and a third reversed primer, the third forward primer, located at the 3′-end region of the forward primer, comprising a fragment having more than 5 nucleotides capable of annealing the third forward primer to the template; and
the third reversed primer having
(a) part (a2), located at the 5′-end region of the third reversed primer, comprising a fragment having more than 10 nucleotides for reversed extending the product obtained in the previous polymerase chain reaction and producing the target polynucleotide; and

(b) part (b2), located at the 3′-end region of the third reversed primer, comprising a fragment having more than 10 nucleotides capable of annealing the third reversed primer to the template,
and wherein the 3′-end of the part (a2) is adjacent to the 5′-end of the part (b2); and
(iii) a fourth set of primer pairs comprising a fourth forward primer and a fourth reversed primer, the fourth forward primer having
(a) part (a3), located at the 5′-end region of the fourth forward primer, comprising a fragment having more than 10 nucleotides for forward extending the product obtained in the previous polymerase chain reaction and producing the target polynucleotide; and
(b) part (b3), located at the 3′-end region of the fourth forward primer, comprising a fragment having more than 10 nucleotides capable of annealing the fourth forward primer to the template,
and wherein the 3′-end of the part (a3) is adjacent to the 5′-end of the part (b3); and
the fourth reversed primer having
(c) part (c3), located at the 5′-end region of the fourth reversed primer, comprising a fragment having more than 10 nucleotides for reversed extending the product obtained in the previous polymerase chain reaction and producing the target polynucleotide; and
(d) part (d3), located at the 3′-end region of the reversed primer, comprising a fragment having more than 10 nucleotides capable of annealing the fourth reversed primer to the template;
and wherein the 3′-end of the part (c3) is adjacent to the 5′-end of the part (d3).

The method according to the invention preferably further comprises adjusting a sequence to change a codon of the target polynucleotide to a codon which has a high expression efficiency in translating a corresponding amino acid in a cell of the host. In another aspect, the invention provides a method for highly expressing a protein encoded by a target polynucleotide in a host, which comprises the steps of:

(1) producing a target polynucleotide obtained by the method for synthesizing a target polynucleotide encoding a protein as described above;

(2) transforming or transfecting the target polynucleotide to the host; and

(3) expressing the target heterogeneous protein in the transformed and transfected host.

### BRIEF DESCRIPTION OF THE DRAWINGS

FIG. 1 illustrates the schematic figure of the method of synthesizing a target polynucleotide according to one embodiment of the invention.

FIG. 2 illustrates the schematic figure of the method of synthesizing a target polynucleotide according to another embodiment of the invention.

FIG. 3 illustrates the result of polymerase chain reaction product subjected to agarose gel electrophoresis when synthesizing the first template according to Example 1 of the invention.

FIG. 4 illustrates the result of polymerase chain reaction product subjected to agarose gel electrophoresis when synthesizing the polynucleotide product encoded by the target polynucleotide according to Example 1 of the invention.

FIG. 5 illustrates the schematic figure of Example 1 of the invention.

FIG. 6 illustrates the construction of PRRSV-ORF7 protein expression vector according to Example 1 of the invention.

FIG. 7 illustrates the expression result of FMD-vpg protein after changing codons subjected to SDS-PAGE according to Example 3 of the invention.

### DETAILED DESCRIPTION OF THE INVENTION

The present invention provides a method of synthesizing a target polynucleotide through a polymerase chain reaction (PCR). Preferably, the target polynucleotide encodes a protein. The method is initiated with a first template of virus genome. The present invention is characterized in that a template that is highly relevant to the target polynucleotide is not necessary. Therefore, the polymerase chain reaction according to the invention can be used to produce various products, even without a template that is highly relevant to the target polynucleotide. Besides, according to the present invention, no purification step is required, and a PCR product obtained in a previous reaction can be directly used in a next reaction.

As used herein, the term “target polynucleotide” refers to a sequence to be produced. A polynucleotide molecule corresponding to the target polynucleotide sequence may not be available or may not even exist in the nature. On the other hand, the target polynucleotide sequence may be a sequence coding for a protein or a peptide, preferably a virus protein.

In one preferred embodiment of the invention, the target polynucleotide encodes a porcine reproductive and respiratory syndrome virus (PRRSV) protein or Taiwanese foot-and-mouth disease (FMD) virus. More preferably, the porcine reproductive and respiratory syndrome virus protein is selected from the group consisting of porcine reproductive and respiratory syndrome virus ORF 1, 2, 3, 4, 5, 6, and 7.

As used herein, the term “host-vector expression system” refers to a system comprising a host organism carrying a vector that contains a coding sequence for expressing a desired protein. Any conventional host organism convenient for maintenance and operation is suitable for practicing the invention. Preferably, the host organism is a microorganism. More preferably, the host organism is an enteric bacterium. The vector has an ability to express the protein in the host. According to the invention, a product can be expressed by a host-vector expression system where the vector is transformed with the target polynucleotide obtained according to the invention.

The method preferably further comprises adjusting a sequence to change a codon of the target polynucleotide to a codon which has a high expression efficiency in translating a corresponding amino acid in a cell of the host cell. In one preferred embodiment of the invention, the target polynucleotide encodes a mutated protein which has multiple mutation sites compared to a wild-type form thereof.

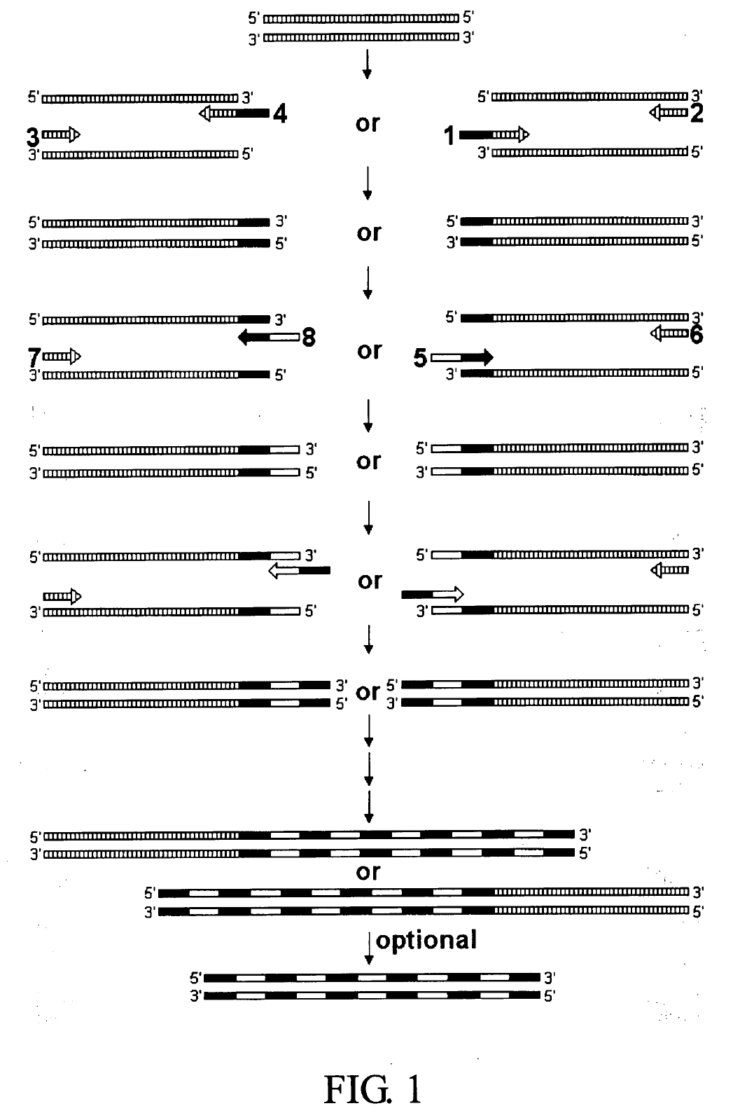

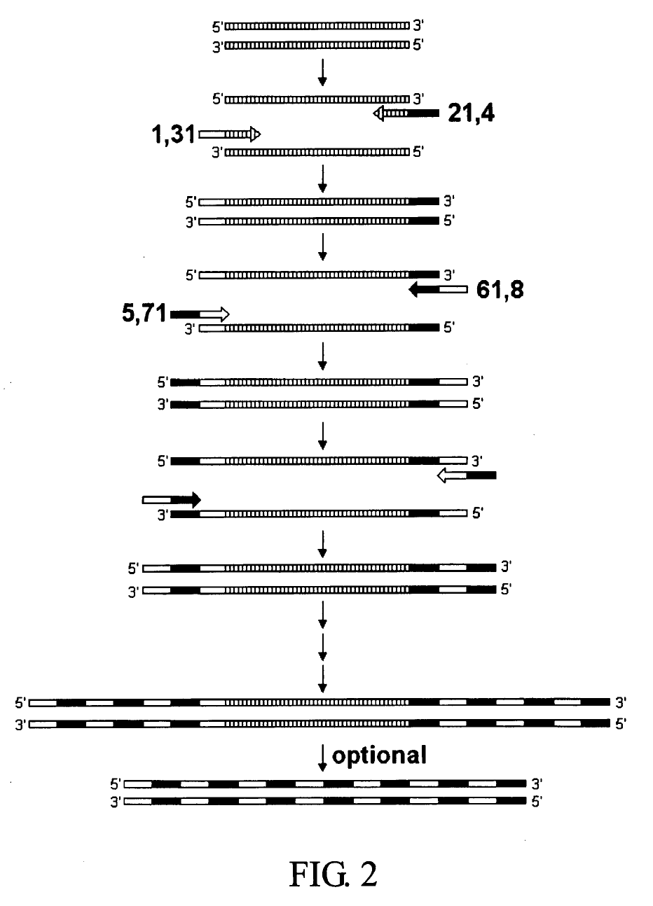

According to the invention, the method comprises conducting a first polymerase chain reaction on a first template with a first set of primer pairs (as shown in FIGS. 1 and 2, primers 1, 2, 3, 4, 21, or 31) to obtain a first polymerase chain reaction (PCR) product. The first template is any template sequence commonly used in the host-vector expression system or a fragment of the target polynucleotide.

As used herein, the term “template” refers to an oligonucleotide fragment used in a polymerase chain reaction for amplifying a molecule, which is the same or highly homologous to the molecule under the conditions ranging from moderate (about 5×SSC at 52° C.) to high (about 0.1×SSC at 65° C.) stringency conditions.

According to the invention, the template sequence commonly used in the host-vector expression system comprises, but is not limited to, a part or whole of a conventional vector, a gene fragment, a promoter fragment, or a polynucleotide fragment containing restriction enzyme recognition sites. In one embodiment of the invention, the first template is a conventional template used in synthesizing a polynucleotide which is not relevant to the target polynucleotide, when the polynucleotide molecule corresponding to the target polynucleotide to be produced does not exist in the nature, or is not available. Preferably, the template applied in the first polymerase chain reaction of the multi-cyclic polymerase chain reaction comprises a polynucleotide fragment encoding a part of the protein. In another aspect, the template applied in the first polymerase chain reaction of the multi-cyclic polymerase chain reaction preferably comprises a polynucleotide fragment irrelevant to the target polynucleotide. As used herein, the polynucleotide fragment irrelevant to the target polynucleotide refers to a polynucleotide fragment having an identity less than 10% compared to the target polynucleotide.

In another aspect, the fragment of the target polynucleotide as the template according to the invention may be a polynucleotide that encodes the protein heterogeneous to the host, where a codon thereof is changed to a codon which has a high expression efficiency in translating a corresponding amino acid in the host cell, or which has multiple mutation sites comparing to a wild-type form thereof.

As used herein, the term “first set of primer pairs” refers to primers comprising an oligonucleotide fragment used for annealing to the first template in the first polymerase chain reaction. In one embodiment of the invention, a helper primer is provided, which is homologous to one primer of the first set of primer pairs and identical to a fragment of the target polynucleotide. The homology between the helper primer and the template may not be enough to lead a successful annealing step. The first set of primer pairs can anneal to the first template for carrying on some cycles in a polymerase chain reaction, and then the helper primer can anneal to a product made by the first set of primer pairs for more cycles of the reaction. Preferably, the amount of the helper primer is more than that of the first set of primer pairs.

According to the invention, the first polymerase chain reaction product provides a starting material for synthesizing the target polynucleotide, and wherein preferably, it also participates in constituting the target polynucleotide.

According to the invention, the method comprises conducting multi-cyclic polymerase chain reactions by a primer extension technique to obtain a product comprising the target polynucleotide; wherein a template used in each of succeeding polymerase chain reaction is a product obtained in a previous polymerase chain reaction, and all of the fragments of the target polynucleotide produced in the polymerase chain reactions in sequence constitute the target polynucleotide sequence. The target polynucleotide is synthesized in fragments during each polymerase chain reaction through un-annealed parts of the primer (as shown in FIGS. 1 and 2, primer 5, 8, 61, or 71) for extension by the primer extension technique. The advantage of the invention is that the product obtained in the previous polymerase chain reaction is directly taken as the template used in the afterward reaction without a purification step or other specific processing steps. The labor and time are less than conventional methods.

According to the invention, the primer pairs used in the polymerase chain reactions are constructed by extending the target polynucleotide at a direction from the 3′-end to the 5′-end (as shown in FIG. 1, right), and/or at a direction from the 5′-end to the 3′-end (as shown in FIG. 1, left). In one preferable embodiment of the invention, the target polynucleotide is extended at two directions, i.e. from the 3′-end to the 5′-end and from the 5′-end to the 3′-end of the target polynucleotide sequence (as shown in FIG. 2).

In one embodiment of the invention, the extension is conducted at the direction from the 3′-end to the 5′-end of the target polynucleotide as shown in FIG. 1, right by using a second set of primer pairs comprising a second forward primer and a second reversed primer.

The second forward primer of the second set of primer pairs is designed to have the following parts:
(a) part (a1), located at the 5′-end region of the second forward primer, comprising a fragment having more than 10 nucleotides for forward extending the product obtained in the previous polymerase chain reaction and producing the target polynucleotide, and
(b) part (b1), located at the 3′-end region of the second forward primer, comprising a fragment having more than 10 nucleotides capable of annealing the second forward primer to the template;
and wherein the 3′-end of the part (a1) is adjacent to the 5′-end of the part (b1).

Briefly, The part (a1) is designed for extending the target polynucleotide, and the part (b1) is for annealing the template enabling DNA polymerase to catalyze DNA synthesis.

The second reversed primer is designed to, located at the 3′-end region of the second reversed primer, comprise a fragment having more than 5 nucleotides capable of annealing to the template. Preferably, the second reversed primer (6) is the same as a first reversed primer (2) of the first set of primer pairs.

In another embodiment of the invention, the extension is conducted at the direction from the 5′-end to the 3′-end of the target polynucleotide as shown in FIG. 1, left by using a third set of primer pairs comprising a third forward primer and a third reversed primer.

The third forward primer of the third set of primer pairs is designed to, located at the 3′-end region, comprise a fragment having more than 5 nucleotides capable of annealing the third forward primer to the template.

The third reversed primer is designed to have the following parts:
(a) part (a2), located at the 5′-end region of the third reversed primer, comprising a fragment having more than 10 nucleotides for reversed extending the product obtained in the previous polymerase chain reaction and producing the target polynucleotide;
(b) part (b2), located at the 3′-end region of the third reversed primer, comprising a fragment having more than 10 nucleotides capable of annealing the third reversed primer to the template,
and wherein the 3′-end of the part (a2) is adjacent to the 5′-end of the part (b2).

Briefly, the part (a2) is designed for extending the target polynucleotide, and the part (b2) is for annealing the template enabling DNA polymerase to catalyze DNA synthesis.

The third forward primer is designed for conducting the polymerase chain reactions. Preferably, the third forward primer (7) used in the method (ii) is the same to a first forward primer (3) of the first set of primer pairs used in the step (1).

In the other embodiment of the invention, the extension is conducted at both directions from the 3′-end to the 5′-end and from the 5′-end to the 3′-end of the target polynucleotide as shown in FIG. 2 by using a fourth set of primer pairs comprising a fourth forward primer and a fourth reversed primer. The fourth forward primer of the fourth set of primer pairs is designed to have the following parts:
(a) part (a3), located at the 5′-end region, comprising a fragment having more than 10 nucleotides for forward the product obtained in the previous polymerase chain reaction and producing the target polynucleotide; and
(b) part (b3), located at the 3′-end region of the fourth forward primer, comprising a fragment having more than 10 nucleotides capable of annealing the fourth forward primer to the template;
and wherein the 3′-end of the part (a3) is adjacent to the 5′-end of the part (b3).

The fourth reversed primer is designed to have the following parts:
(c) part (c3), located at the 5′-end region, comprising a fragment having more than 10 nucleotides for reversed extending the product obtained in the previous polymerase chain reaction and producing the target polynucleotide;
(d) part (d3), located at the 3-end region of the fourth reversed primer, comprising a fragment having more than 10 nucleotides capable of annealing the fourth reversed primer to the template;
and wherein the 3′-end of the part (c3) is adjacent to the 5′-end of the part (d3).

The parts (a3) and (c3) are designed for extending the target polynucleotide, and the parts (b3) and (d3) are designed for annealing the template enabling DNA polymerase to catalyze DNA synthesis. If the first template is irrelevant to the target polynucleotide, the 3′-end of the part (a3) and the 3′-end of the part (c3) are designed to be adjacent to each other in the target polynucleotide. For the reason, the target polynucleotide can be generated without disruption or discontinuation by the removal of the first template. If the first template is a fragment of the target polynucleotide, the 3′-end of the part (a3) is designed to be adjacent to the 5′-end of the first template in the target polynucleotide, and the 3′-end of the part (c3) is designed to be adjacent to the 3′-end of the first template in the target polynucleotide for constituting the target polynucleotide.

According to the invention, the primer sequences can be easily determined by artisans skilled in this field, and the conditions and the step of conducting the multi-cyclic polymerase chain reaction can be designed by artisans skilled in this field. Preferably, each primer used in each step is a fragment having more than 10 nucleotides, most preferably more than 15 nucleotides.

According to the invention, the method preferably further comprises obtaining a polynucleotide product comprising the target polynucleotide from a final product of the multi-cyclic polymerase chain reactions.

According to the invention, the method preferably further comprises a step of removing the nucleotide sequence of the first template from the final product so as to obtain the target polynucleotide if the first template is irrelevant to the target polynucleotide, such as the template sequence commonly used in the host-vector expression system. It can be achieved by an enzyme-digesting step or other conventional methods. Preferably, the first template is designed to have restriction enzyme recognition sites at both ends, which are convenient to manipulation hereafter.

In one preferred embodiment of the invention, the multi-cyclic polymerase chain reactions are conducted with primers selected from the group consisting of the first, second, third, and fourth set of primer pairs.

Preferably, the second set of primer pairs consist of the second forward primer and the second reversed primer; the third set of primer pairs consist of the third forward primer and the third reversed primer; and the fourth set of primer pairs consist of the fourth forward primer and the fourth reversed primer, respectively.

For easy manipulation, the primers in the multi-cyclic polymerase chain reactions according to invention preferably comprise no more than 50 nucleotides.

In one preferred embodiment of the invention, the length of the 3′-portion of the primers of the multi-cyclic polymerase chain reactions is larger than 15 nucleotides; and the length of the 5′-portion of the primers of the multi-cyclic polymerase chain reactions is larger than 15 nucleotides, respectively.

In one preferred embodiment of the invention, each of the multi-cyclic polymerase chain reactions is conducted only on a template that consists of a product that has been extended with the addition of nucleotides in a previous polymerase chain reaction, whereby the product of each succeeding polymerase chain reaction is longer than the product of each previous polymerase chain reaction.

The present invention also provides a method for highly expressing a protein encoded by a target polynucleotide in a host, which comprises the steps of:

(1) producing a target polynucleotide obtained by the method for synthesizing a target polynucleotide as described above;

(2) transforming or transfecting the target polynucleotide to the host; and

(3) expressing the target heterogeneous protein in the transformed and transfected host.

According to the invention, the method preferably further comprises adjusting a sequence of the one or more sets of primer pairs to change fragments of the target polynucleotide by changing a codon according to the data provided in the Wisconsin Package (The Wisconsin Package, by Genetics Computer Group, Inc. (1992)). For instance, the codon CTA encoding leucine may be changed to CTG, CTT, CTC, TTG, or TTA; the codon ATA encoding isoleucine may be changed to ATC or ATT; the codons CGG, AGG, AGA encoding arginine may be changed to CGT or CGC; the codon GGA encoding glycine may be changed to GGT or GGC; the codon CCC encoding proline may be changed to CCG, CCA or CCT; the codon CTA encoding leucine may be changed to CTG, CTT, CTC, TTG, or TTA; the codon ATA encoding isoleucine may be changed to ATC or ATT; the codons CGG, AGG, AGA encoding arginine may be changed to CGT or CGC; the codon GGA encoding glycine may be changed to GGT or GGC; or the codon CCC encoding proline may be changed to CCG, CCA or CCT, in order to enhance the translation rate.

In one embodiment of the invention, the method of transforming or transfecting the target polynucleotide to the host used in the step (2) may be any conventional method for introducing the polynucleotide into the host. Preferably, the polynucleotide having the target sequence is incorporated into a vector.

In one embodiment of the invention, the conditions for highly expressing the protein in the transformed or transfected host used in the step (3) may be determined by artisans skilled in this field according to the properties of the heterogeneous protein and the host cell.

According to the invention, a heterogeneous protein can be translated by the target polynucleotide in a host; therefore, the problem of the low yield of expressing a heterogeneous protein in a host is solved.

The following Examples are given for the purpose of illustration only and are not intended to limit the scope of the present invention.

### EXAMPLE 1

### Method I for Synthesizing a Target Polynucleotide Encoding PRRSV-ORF7 Protein Efficiently Expressed in Escherichia coli

Target polynucleotide: PRRSV-ORF 7 is a gene encoding a nucleocapsid protein in porcine reproductive and respiratory syndrome virus (PRRSV), and the sequence of the gene was obtained from the National Center Biotechnology Information. In the sequence, the codon CTA encoding leucine was changed to CTG, CTT, CTC, TTG, or TTA; the codon ATA encoding isoleucine to ATC or ATT, the codons CGG, AGG, AGA encoding arginine to CGT or CGC; the codon GGA encoding glycine to GGT pr GGC; and the codon CCC encoding proline to CCG, CCA or CCT according to the table of the codons for a high expression in E. coli in the Wisconsin Package. The changes of the codons were listed in Table 1.

A gene encoding PRRSV-ORF 7 in PRRSV was then designed as a target polynucleotide sequence as shown in SEQ ID NO: 1 for highly expressed in E. coli.

TABLE 1

| Aa | Codon | Number¹ | /1000² | Fraction³ |
|---|---|---|---|---|
| Gly | GGG | 13 | 1.89 | 0.02 |
| Gly | GGA | 3 | 0.44 | 0.00 |
| Gly | GGU | 365 | 52.99 | 0.59 |
| Gly | GGC | 238 | 34.55 | 0.38 |
| Glu | GAG | 108 | 15.68 | 0.22 |
| Glu | GAA | 394 | 57.20 | 0.78 |
| Asp | GAU | 149 | 21.63 | 0.33 |
| Asp | GAC | 298 | 43.26 | 0.67 |
| Val | GUG | 93 | 13.50 | 0.16 |
| Val | GUA | 146 | 21.20 | 0.26 |
| Val | GUU | 289 | 43.26 | 0.51 |
| Val | GUC | 38 | 5.52 | 0.07 |
| Ala | GCG | 161 | 23.37 | 0.26 |
| Ala | GCA | 173 | 25.12 | 0.28 |
| Ala | GCU | 212 | 30.78 | 0.35 |
| Ala | GCC | 62 | 9.00 | 0.10 |
| Arg | AGG | 1 | 0.15 | 0.00 |
| Arg | AGA | 0 | 0.00 | 0.00 |
| Ser | AGU | 9 | 1.31 | 0.03 |
| Ser | AGC | 71 | 10.31 | 0.20 |
| Lys | AAG | 111 | 16.11 | 0.26 |
| Lys | AAA | 320 | 46.46 | 0.74 |
| Asn | AAU | 19 | 2.76 | 0.06 |
| Asn | AAC | 274 | 39.78 | 0.94 |
| Met | AUG | 170 | 24.68 | 1.00 |
| Ile | AUA | 1 | 0.15 | 0.00 |
| Ile | AUU | 70 | 10.16 | 0.17 |
| Ile | AUC | 345 | 50.09 | 0.83 |
| Thr | ACG | 25 | 3.63 | 0.07 |
| Thr | ACA | 14 | 2.03 | 0.04 |
| Thr | ACU | 130 | 18.87 | 0.35 |
| Thr | ACC | 206 | 29.91 | 0.55 |
| Trp | UGG | 55 | 7.98 | 1.00 |
| Stop | UGA | 0 | 0.00 | (Stop) |
| Cys | UGU | 22 | 3.19 | 0.49 |
| Cys | UGC | 23 | 3.34 | 0.51 |
| Stop | UAG | 0 | 0.00 | (Stop) |
| Stop | UAA | 0 | 0.00 | (Stop) |
| Tyr | UAU | 51 | 7.4 | 0.25 |
| Tyr | UAC | 157 | 22.79 | 0.75 |
| Leu | UUG | 18 | 2.61 | 0.03 |
| Leu | UUA | 12 | 1.74 | 0.02 |
| Phe | UUU | 51 | 7.4 | 0.24 |
| Phe | UUC | 166 | 24.10 | 0.76 |
| Ser | UCG | 14 | 2.03 | 0.04 |
| Ser | UCA | 7 | 1.02 | 0.02 |
| Ser | UCU | 120 | 17.42 | 0.34 |
| Ser | UCC | 131 | 19.02 | 0.37 |
| Arg | CGG | 1 | 0.15 | 0.00 |
| Arg | CGA | 2 | 0.29 | 0.01 |
| Arg | CGU | 290 | 42.10 | 0.74 |
| Arg | CGC | 96 | 13.94 | 0.25 |
| Gln | CAG | 233 | 33.83 | 0.86 |
| Gln | CAA | 37 | 5.37 | 0.14 |
| His | CAU | 18 | 2.61 | 0.17 |
| His | CAC | 85 | 12.34 | 0.83 |
| Leu | CUG | 480 | 69.69 | 0.83 |
| Leu | CUA | 2 | 0.29 | 0.00 |
| Leu | CUU | 25 | 3.63 | 0.04 |
| Leu | CUC | 38 | 5.52 | 0.07 |
| Pro | CCG | 190 | 27.58 | 0.77 |
| Pro | CCA | 36 | 5.23 | 0.15 |
| Pro | CCU | 19 | 2.76 | 0.08 |
| Pro | CCC | 1 | 0.15 | 0.00 |

¹Number of occurrences of the codon in the genes from which the table is compiled.
²Expected number of occurrences per 1000 codon in genes whose codon usage is identical to that compiled in the frequency table.
³Fraction of occurrences of the codon in synonymous codon family.

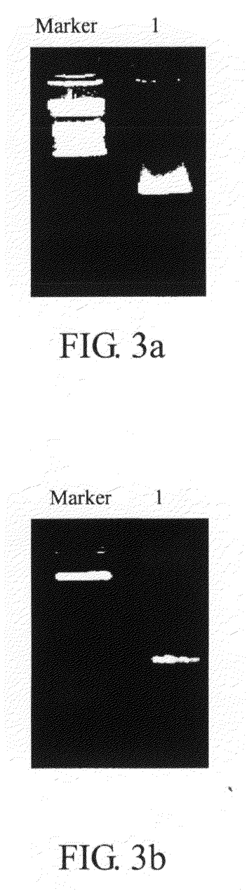

First Template: The wild type PRRSV genome was taken for generating the first template. In order to clone the target polynucleotide sequence (SEQ ID NO: 1) in an expression plasmid, pET23a, a forward primer, ORF7-pET23a-Nde I-F, 5′-CCGCGCGGCAGCCATATGCCAAATAACAAC-3′ (SEQ ID NO: 2) comprising a cloning site in its 5′-portion and first 18 nucleotides of SEQ ID NO: 1 in its 3′-portion along with a reversed primer, ORF7-C-R0, 5′-CTTCTTATTTTTACTACCCGGTCCCTTAACTCTGGA-3′ (SEQ ID NO: 3) for changing the codons GGA and AGG to GGT and AGT were used. The polymerase chain reaction was carried out with adding Pfu polymerase, dNTP and reaction buffer. The thermocycle was 5 minutes at 95° C. followed by 20 cycles of 1 minute at 94° C., 30 seconds at 55° C. and 1 minute at 72° C. The product was subject to agarose gel electrophoresis. The result was shown in FIG. 3a, lane 1. The product was further taken together with the forward primer, ORF7-pET23a Nde I-F (SEQ ID NO: 2) and a reversed primer ORF7-C-R1, 5′-CTTCTTATTTTTACGACCCGGACCCTTAACACGGGA-3′ (SEQ ID NO: 4) to carry out another polymerase chain reaction as described above. In the ORF7-C-R1, the codons AGA and GGA to CGT and GGT were changed. The product obtained was used as the first template and the result of agarose gel electrophoresis was shown in FIG. 3b, lane 1.

First Set of Primer Pairs: The extension was from the 5′-end to the 3′-end of the target sequence. The forward primer ORF7-pET23a-Nde I-F (SEQ ID NO: 2) and a reversed primer ORF7-C-R2,5′-TGCGGCTTCTCCGGGTTTTTCTTCTTATTTTTACG-3′ (SEQ ID NO: 5) were as the first set of primer pairs. The 3′-portion of ORF7-C-R2 was used for annealing 129 to 141 nt of SEQ ID NO: 1, and the 5′-portion was for generating the 142 to 164 nt of SEQ ID NO: 1.

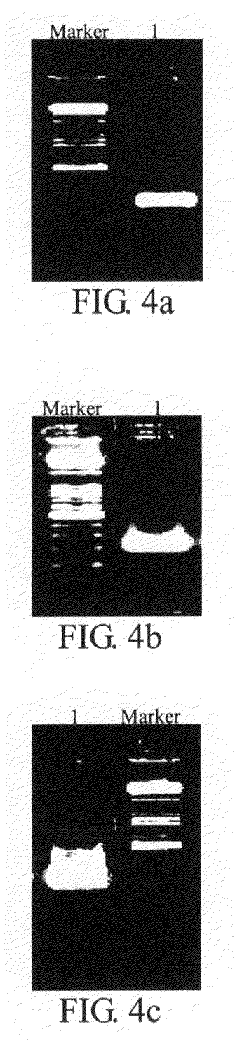

First Polymerase Chain Reaction Product: One μL of the first template, 4 μL of each of the first set of primer pairs, dNTP, reaction buffer, and Pfu polymerase were mixed to conduct a multi-cyclic polymerase chain reaction. The thermocycle was 5 minutes at 95° C. followed by 20 cycles of 1 minute at 94° C., 30 seconds at 55° C. and 1 minute at 72° C. The product was subject to agarose gel electrophoresis. The result was shown in FIG. 4a, lane 1.

Third Set Primer Pairs: The forward primer ORF7-pET23a-Nde I-F (SEQ ID NO: 2) and a reversed primer ORF7-C-R3,5′-GTCGCCAGAGGAAAATGCGGCTTCTCCGGGTTT-3′ (SEQ ID NO: 6) were used as a second primer pair. The (b2) part (3′-end region) of ORF7-C-R3 was used for annealing to the first polymerase chain reaction product as shown in 147 to 164 nt of SEQ ID NO: 1, and the (a2) part (5′-end region) was for generating the 165 to 179 nt of SEQ ID NO: 1.

Polymerase Chain Reaction Product: One μL of the first polymerase chain reaction product, 4 μL of each of the second primer pair, dNTP, reaction buffer, and Pfu polymerase were mixed to conduct a multi-cyclic polymerase chain reaction. The thermocycle was 5 minutes at 95° C. followed by 20 cycles of 1 minute at 94° C., 30 seconds at 55° C. and 1 minute at 72° C. The product was subjected to agarose gel electrophoresis. The result was shown in FIG. 4b, lane 1.

Third Set of Primer Pairs: The forward primer ORF7-pET23a-Nde I-F (SEQ ID NO: 2) and a reversed primer ORF7-C-R4, 5′-TGGTGACGGACGTCATCTTCAGTCGCCAGAGG-3′ (SEQ ID NO: 7) were as the third primer pair. The (b2) part (3′-end region) of ORF7-C-R4 was used for annealing the second polymerase chain reaction product as shown in 169 to 179 nt of SEQ ID NO: 1, and the (a2) part (5′-end region) was used for generating the 180 to 200 nt of SEQ ID NO: 1.

Polymerase Chain Reaction Product: One μL of the polymerase chain reaction product of the third second primer pair, 4 μL of each of the new second primer pair, dNTP, reaction buffer, and Pfu polymerase were mixed to conduct a multi-cyclic polymerase chain reaction. The thermocycle was 5 minutes at 95° C. followed by 20 cycles of 1 minute at 94° C., 30 seconds at 55° C. and 1 minute at 72° C. The product was subjected to sequence analysis and agarose gel electrophoresis. The electrophoresis result was shown in FIG. 4c, lane 1. Therefore, the target polynucleotide whose sequence as shown in SEQ ID NO: 1 was obtained.

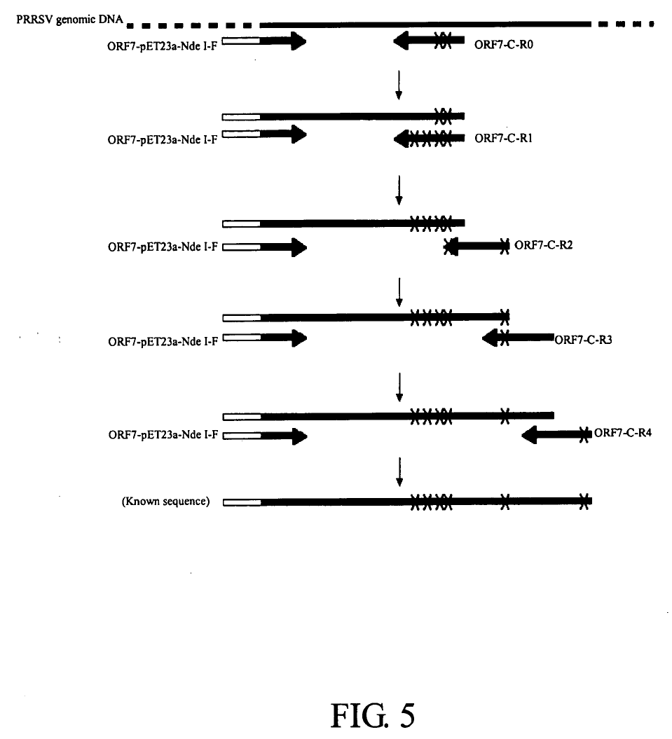

The schematic figure of the example was shown in FIG. 5.

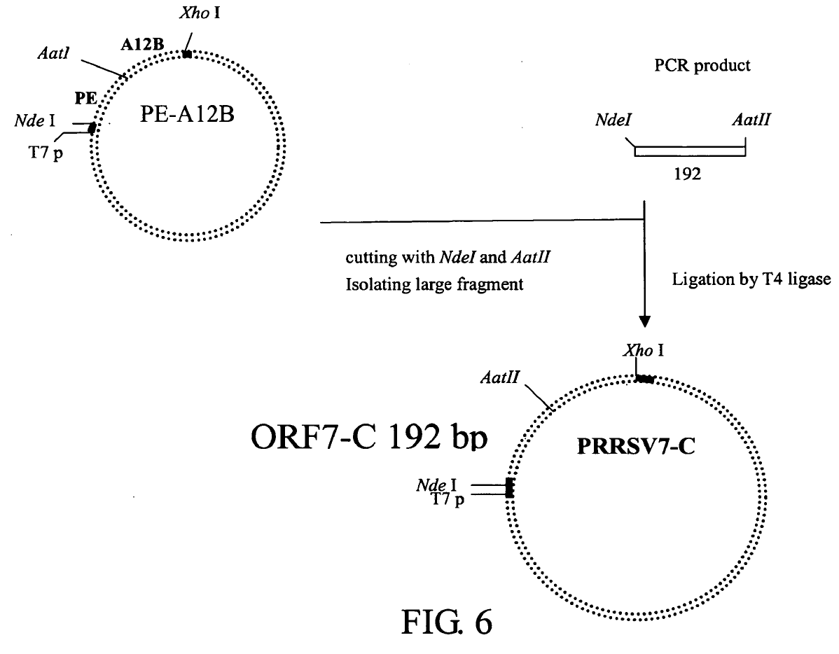

Cloning: The polymerase chain reaction product and PE-A12B plasmid with a backbone of pET23a were both digested with restriction enzymes Nde I and Aat II. The larger fragments were taken and ligated together to obtain the plasmid PPRSV7-C by T4 ligase (as shown in FIG. 6).

Expressing: The PRRSV7-C plasmid was transformed to JM109 competent cells for expression.

### EXAMPLE 2

### Method II for Synthesizing a Target Polynucleotide Encoding PRRSV-ORF7 Protein Efficiently Expressed in Escherichia coli

Example 2 provides a method for synthesizing PRRSV-ORF7, which is similar to Example 1 with some modifications.

The target polynucleotide sequence (SEQ ID NO: 1) in the example was as described in Example 1, and the first template was a part of the wild type PRRSV genome.

First Set of Primer Pairs: The first set of primer pairs used in the example was ORF7-pET23a-Nde I-F (SEQ ID NO: 2) and ORF7-C-R0 (SEQ ID NO: 3). A helper primer ORF7-C-R1 (SEQ ID NO: 4) was also provided. ORF7-C-R0 and ORF7-C-R1 were in the ratio of 1:19. The 3′-end region of ORF7-C—R0 was used for annealing the first template, and the 5′-end region was for generating a part of the target polynucleotide sequence shown in SEQ ID NO: 1. The 3′-end region of ORF7-C-R1 was also part of SEQ ID NO: 1 and can anneal the product made by the ORF7-pET23a-Nde I-F and ORF7-C-R0.

First Polymerase Chain Reaction Product: One μL of the adapter template polymerase, 4 μL of ORF7-pET23a-Nde I-F, 0.2 μL of ORF7-C-R0, 3.8 μL of ORF7-C-R1, dNTP, reaction buffer, and Pfu polymerase were mixed to conduct a multi-cyclic polymerase chain reaction. The thermocycle was 5 minutes at 95° C. followed by 20 cycles of 1 minute at 94° C., 30 seconds at 55° C. and 1 minute at 72° C. The product was subject to agarose gel electrophoresis. The product was as the first template in Example 1 and the steps hereafter of synthesizing PRRSV-ORF7 were similar to the description in Example 1.

### EXAMPLE 3

### Method of Synthesizing a Target Polynucleotide Encoding FMD-vpg Protein Efficiently Expressed in E. coli

Target Polynucleotide: FMD-vpg (3828-5975) was a gene encoding a non-structural protein of Taiwanese foot-and-mouth disease (FMD) virus, and the sequence of the gene was reported by Beard et al. (Beard, C. W. and Mason, P. W. 2000. Genetic determinants of altered virulence of Taiwanese foot-and-mouth disease virus J. Virol 74 (2), 987-991). In the FMD-vpg, the codon GGA encoding glycine was changed to GGT; the codon AGA encoding leucine to CGT; and the codon ATA encoding isoleucine to ATC to enhance the expression of the protein in an enteric bacterium (see Table 1). A gene encoding FMD-vpg in Taiwanese foot-and-mouth disease virus was then designed to a target polynucleotide sequence as shown in SEQ ID NO: 8 for highly expressed in E. coli.

First Template: A part of pET-23a (SEQ ID NO: 9), which was a template sequence commonly used in the host-vector expression system, was taken as the first template, where the sequence was known and had cloning sites for manipulation.

First Set of Primer Pairs: The extension was toward the 5′-end of the SEQ ID NO: 10. 3B-F1, 5′-TTGATCGTCACTGAGGTCGACAAGCTTGCG-3′ (SEQ ID NO: 10) and a reversed primer T7t-R1,5′-TTATGCTAGTTATTGCTCAGCGGTGGCAGC-3′ (SEQ ID NO: 11) were as the first set of primer pairs. The 3′-end region of 3B-F1 was used for annealing the 1 to 15 nt of SEQ ID NO: 9, and the 5′-end region was for generating the 148 to 162 nt of SEQ ID NO: 8.

First Polymerase Chain Reaction Product: One μL of the first template, 4 μL of each of the first set of primer pairs, dNTP, reaction buffer, and Pfu polymerase were mixed to conduct a multi-cyclic polymerase chain reaction. The thermocycle was 5 minutes at 95° C. followed by 20 cycles of 1 minute at 94° C., 30 seconds at 55° C. and 1 minute at 72° C.

Second Set of Primer Pairs: T7t-R1 (SEQ ID NO: 11) and a forward primer 3B-F2,5′-AGTGAAAGCAAAGAACTTGATCGTCACTGAG-3′ (SEQ ID NO: 12) were as the second primer pair. The (b1) part (3′-end region) of 3B-F2 was used for annealing the first polymerase chain reaction product as 148 to 162 nt of SEQ ID NO: 8, and the (a1) part (5′-end region) was for generating the 132 to 147 nt of SEQ ID NO: 8.

Polymerase Chain Reaction Product: One μL of the second polymerase chain reaction product, 4 μL of each of the second primer pair, dNTP, reaction buffer, and Pfu polymerase were mixed to conduct a multi-cyclic polymerase chain reaction. The thermocycle was 5 minutes at 95° C. followed by 20 cycles of 1 minute at 94° C., 30 seconds at 55° C. and 1 minute at 72° C.

Primer Pair: T7t-R1 (SEQ ID NO: 11) and series of forward primers were used as the primer pair sequentially:

3B-F3:

(SEQ ID NO: 13)

5′-ACCTGTCGCTTTGAAAGTGAAAGCAAAGAAC-3′;
3B-F4:

(SEQ ID NO: 14)

5′-GGTCCGGTGAAGAAACCTGTCGCTTTGAAA-3′;
3B-F5:

(SEQ ID NO: 15)

5′-GAAGGTCCTTACGAGGGTCCGGTGAAGAAA-3′;
3B-F6:

(SEQ ID NO: 16)

5′-AAAGCCCCGGTCGTGAAGGAAGGTCCTTACGAG-3′;
3B-F7;

(SEQ ID NO: 17)

5′-ACCGCTGAAGGTGAAAGCAAAAGCCCCGGTCGTG-3′;
3B-F8:

(SEQ ID NO: 18)

5′-CCAATGGAGCGTCAGAAACCGCTGAAGGTGAAA-3′;
3B-F9;

(SEQ ID NO: 19)

5′-GAGGGTCCATACGCCGGCCCAATGGAGCGTCAGA-3′;
3B-F10;

(SEQ ID NO: 20)

5′-AAAAAATCCCATATGGAGGGTCCATACGCC-3′.

The (b1) parts (3′-end regions) of the 3B-F3 to 3B-F10 were used for annealing the former polymerase chain reaction product, and the (a1) parts (5′-end regions) were used for generating the polynucleotide whose sequence was shown in SEQ ID NO: 8. The condition of conducting the polymerase chain reaction was similar to the condition used for conducting the first chain reaction product.

Cloning: The polymerase chain reaction product and an IPTG inductive expression plasmid were both digested with restriction enzymes Xho I and Nde I. The larger fragments were taken and ligated together by T4 ligase.

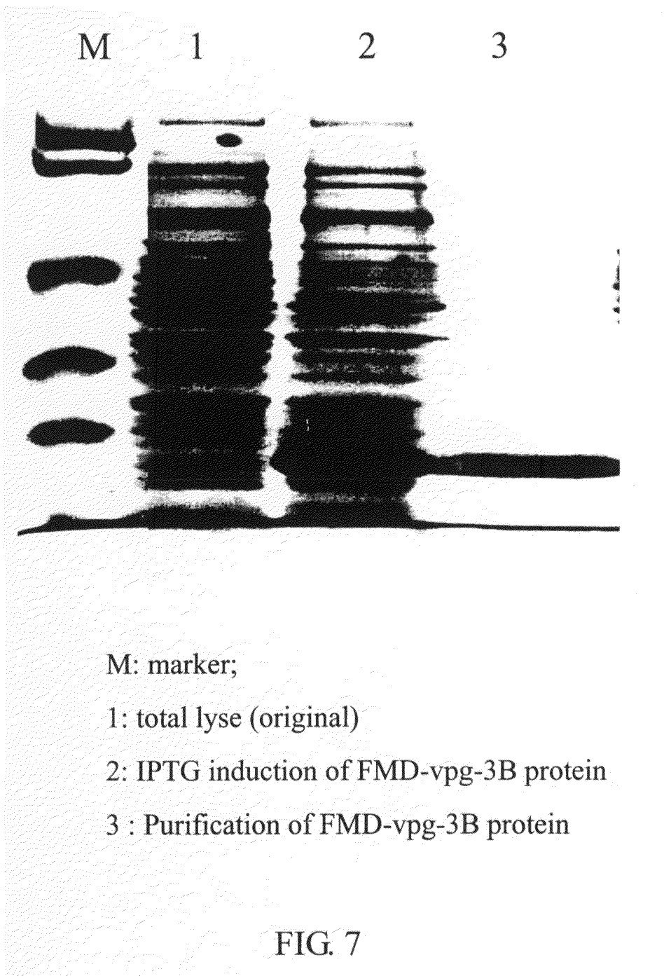

Expression: The vector obtained was transformed to JM109 competent cells for expression. FMD-vpg-3B protein was expressed and purified for resolving in SDS-PAGE and shown in FIG. 7. It showed that the protein with some codon changes could be expressed in a high level with the induction of IPTG.

### EXAMPLE 4

### Primer Length for Carrying Out Polymerase Chain Reaction

Primers with different lengths were taken for carrying out polymerase chain reaction. The primer design and method for polymerase chain reaction were similar to those in Example 1. The primer pair was listed in Table 2.

TABLE 2

| Primer | Length | Length of | Length of |
|---|---|---|---|
| pair | of primer | 3′-end region | 5′-end region |
| 1 | 16 | 8 | 8 |
| 2 | 20 | 10 | 10 |
| 3 | 24 | 12 | 12 |
| 4 | 30 | 15 | 15 |
| 5 | 25 | 10 | 15 |

The result of polymerase chain reaction was shown in Table 3, wherein “+” refers to that a desired fragment was observed in an agarose gel electrophoresis; “−” refers to that a desired fragment was not observed in an agarose gel electrophoresis. It showed that the lengths of the 3′-end region and the 5′-end region should be both more than 10 nucleotides.

TABLE 3

| | Primer pair | | | | |
|---|---|---|---|---|---|
| | 1 | 2 | 3 | 4 | 5 |
| Result | − | − | + | + | + |

While embodiments of the present invention have been illustrated and described, various modifications and improvements can be made by persons skilled in the art. The embodiments of the present invention are therefore described in an illustrative but not restrictive sense. It is intended that the present invention is not limited to the particular forms as illustrated, and that all the modifications not departing from the spirit and scope of the present invention are within the scope as defined in the appended claims.

What is claimed is:

1. A method for synthesizing a target polynucleotide encoding a protein, comprising conducting multi-cyclic polymerase chain reactions by a primer extension technique to obtain a product comprising the target polynucleotide; wherein a first polymerase chain reaction of the multi-cyclic polymerase chain reactions is conducted on a template and with a first set of primer pairs, and succeeding polymerase chain reactions are conducted on a template that is a product obtained in a previous polymerase chain reaction; and the succeeding polymerase chain reactions are conducted with one or more sets of primer pairs comprising:
(i) a second set of primer pairs comprising a second forward primer and a second reversed primer, the second forward primer having two parts:
(a) part (a1), located at the 5′-end region of the second forward primer, comprising a fragment having more than 10 nucleotides for forward extending the product obtained in the previous polymerase chain reaction and producing the target polynucleotide, and
(b) part (b1), located at the 3′-end region of the second forward primer, comprising a fragment having more than 10 nucleotides capable of annealing the second forward primer to the template;
and wherein the 3′-end of the part (a1) is adjacent to the 5′-end of the part (b1); and
the second reversed primer, located at the 3′-end region of the second reversed primer, comprising a fragment having more than 5 nucleotides capable of annealing the second reversed primer to the template;
(ii) a third set of primer pairs comprising a third forward primer and a third reversed primer, the third forward primer, located at the 3′-end region of the third forward primer, comprising a fragment having more than 5 nucleotides capable of annealing the third forward primer to the template; and
the third reversed primer having
(a) part (a2), located at the 5′-end region of the third reversed primer, comprising a fragment having more than 10 nucleotides for reversed extending the product obtained in the previous polymerase chain reaction and producing the target polynucleotide; and
(b) part (b2), located at the 3′-end region of the third reversed primer, comprising a fragment having more than 10 nucleotides capable of annealing the third reversed primer to the template,
and wherein the 3′-end of the part (a2) is adjacent to the 5′-end of the part (b2); and
(iii) a fourth set of primer pairs comprising a fourth forward primer and a fourth reversed primer, the forward primer having
(a) part (a3), located at the 5′-end region of the fourth forward primer, comprising a fragment having more than 10 nucleotides for forward extending the product obtained in the previous polymerase chain reaction and producing the target polynucleotide; and
(b) part (b3), located at the 3′-end region of the fourth forward primer, comprising a fragment having more than 10 nucleotides capable of annealing the fourth forward primer to the template, and wherein the 3′-end of the part (a3) is adjacent to the 5′-end of the part (b3); and
the fourth reversed primer having
(c) part (c3), located at the 5′-end region of the fourth reversed primer, comprising a fragment having more than 10 nucleotides for reversed extending the product obtained in the previous polymerase chain reaction and producing the target polynucleotide; and
(d) part (d3), located at the 3′-end region of the fourth reversed primer, comprising a fragment having more than 10 nucleotides capable of annealing the fourth reversed primer to the template;
and wherein the 3′-end of the part (c3) is adjacent to the 5′-end of the part (d3).

2. The method according to claim 1, wherein the template applied in the first polymerase chain reaction of the multi-cyclic polymerase chain reaction comprises a polynucleotide fragment encoding a part of the protein.

3. The method according to claim 1, wherein the template applied in the first polymerase chain reaction of the multi-cyclic polymerase chain reaction comprises a polynucleotide fragment irrelevant to the target polynucleotide.

4. The method according to claim 1, further comprising adjusting a sequence of the one or more sets of primer pairs to change a codon of the target polynucleotide to a codon, which has a high expression efficiency in translating a corresponding amino acid in a cell of a host.

5. The method according to claim 4, wherein the host is an enteric bacterium.

6. The method according to claim 1, wherein the protein is a virus protein.

7. The method according to claim 6, wherein the virus is a porcine reproductive and respiratory syndrome virus (PRRSV) or Taiwanese foot-and-mouth disease (FMD) virus.

8. The method according to claim 1, wherein the target polynucleotide encodes a mutated protein, which has multiple mutation sites compared to a wild-type form thereof.

9. The method according to claim 1, wherein the first polymerase chain reaction of the multi-cyclic polymerase chain reactions is further conducted by a helper primer, which is homologous to one primer of the first set of primer pairs and identical to a fragment of the target polynucleotide.

10. The method according to claim 1, wherein the multi-cyclic polymerase chain reactions are conducted with primers selected from the group consisting of the first, second, third, and fourth set of primer pairs.

11. The method according to claim 1, wherein the second set of primer pairs consist of the second forward primer and the second reversed primer.

12. The method according to claim 1, wherein the third set of primer pairs consist of the third forward primer and the third reversed primer.

13. The method according to claim 1, wherein the fourth set of primer pairs consist of the fourth forward primer and the fourth reversed primer.

14. The method according to claim 1, wherein the primers of the multi-cyclic polymerase chain reactions comprise no more than 50 nucleotides.

15. The method according to claim 1, wherein the length of the 3′-portion of the primers of the multi-cyclic polymerase chain reactions is larger than 15 nucleotides.

16. The method according to claim 1, wherein the length of the 5′-portion of the primers of the multi-cyclic polymerase chain reactions is larger than 15 nucleotides.

17. The method according to claim 1, wherein each of the multi-cyclic polymerase chain reactions is conducted only on a template that consists of a product that has been extended with the addition of nucleotides in a previous polymerase chain reaction whereby the product of each succeeding polymerase chain reaction is longer than the product of each previous polymerase chain reaction.

18. A method for highly expressing a protein encoded by a target polynucleotide in a host, which comprises the steps of:
(1) producing a target polynucleotide obtained by the method according to claim 1;
(2) transforming or transfecting the target polynucleotide to the host; and
(3) expressing the target heterogeneous protein in the transformed or transfected host.

19. The method according to claim 18, wherein the host is an enteric bacterium.

20. The method according to claim 19, which further comprises, adjusting a sequence of the one or more sets of primer pairs to change fragments of the target polynucleotide, by changing the codon CTA encoding leucine to CTG, CTT, CTC, TTG, or TTA; the codon ATA encoding isoleucine to ATC or ATT; the codons CGG, AGG, AGA encoding arginine to CGT or CGC; the codon GGA encoding glycine to GGT or GGC; the codon CCC encoding proline to CCG, CCA or CCT; the codon CTA encoding leucine to CTG, CTT, CTC, TTG, or TTA; the codon ATA encoding isoleucine to ATC or ATT; the codons CGG, AGG, AGA encoding arginine to CGT or CGC; the codon GGA encoding glycine to GGT or GGC; or the codon CCC encoding proline to CCG, CCA or CCT.
